(19) United States
(12) Patent Application Publication
(10) Pub. No.: US 2026/0091226 A1
(43) Pub. Date: Apr. 2, 2026

(54) TRANSDUCER ARRAY GENERATING TUMOR TREATING FIELD AND METHODS OF PRODUCTION AND USE THEREOF

(71) Applicant: Novocure GmbH, Baar (CH)

(72) Inventor: Richard Deslauriers, Woodbury, CT (US)

(21) Appl. No.: 19/345,507

(22) Filed: Sep. 30, 2025

Related U.S. Application Data
(60) Provisional application No. 63/701,235, filed on Sep. 30, 2024.

(51) Int. Cl. A61N 1/36; A61N 1/04

(52) U.S. Cl. CPC A61N 1/36002; A61N 1/0488

(57) ABSTRACT

A transducer array, comprising an electrode assembly, operable to supply an electrical signal having an alternating current waveform at a frequency in a range from 50 kHz to 1 MHz, and a conductive skin adjacent layer attached to an electrically conductive layer. The electrode assembly comprises a substrate; an electrically conductive layer; and a movable connector extending between a substrate and the electrically conductive layer. The movable connector is constructed of an electrically conductive material and movable so as to enable movement of the electrically conductive layer relative to the substrate so as to permit movement of the electrically conductive layer responsive to movement of the patient's skin. The conductive skin adjacent layer provides an electrical interface between the electrically conductive layer and the patient's skin.

### CROSS-REFERENCE TO RELATED APPLICATIONS

This application claims benefit under 35 U.S.C. § 119(e) of the provisional patent application identified by U.S. Ser. No. 63/701,235, filed Sep. 30, 2024, the entire content of which is hereby expressly incorporated herein by reference.

### BACKGROUND

The following disclosure generally relates to a transducer array with improved mobility and electrical contact with the skin of a patient during Tumor Treating Fields (TTFields or TTFs) therapy, also known as alternating electric field therapy.

Tumor Treating Fields (TTFields or TTFs) are low intensity (e.g., 1-3 V/cm) alternating electric fields within the intermediate frequency range (50 kHz-1 MHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776, which are expressly incorporated herein by reference in their entirety. TTFields are typically delivered through two pairs of transducer arrays that generate perpendicular fields within the treated tumor; the transducer arrays that make up each of these pairs are positioned on opposite sides of the body part that is being treated. More specifically, for the OPTUNE® system manufactured by Novocure GmbH, whose global headquarters is located at Neuhofstrasse 21, 6340 Baar, Switzerland, one pair of electrodes of the transducer array is located to the left and right (LR) of the tumor, and the other pair of electrodes of the transducer array is located anterior and posterior (AP) to the tumor.

The TTFields therapy has been shown to trigger a reduction in tumor mass over time and to ultimately result in improved patient outcomes. TTFields are approved for the treatment of glioblastoma multiforme (GBM), and may be delivered, for example, via the OPTUNE® system, which includes transducer arrays placed on the patient's shaved head. More recently, TTFields therapy has been approved as a combination therapy with chemotherapy for malignant pleural mesothelioma (MPM), and may find use in treating tumors in other parts of the body.

One approach to applying the TTField in different directions is to apply the field between a first set of electrodes for a period of time, then applying a field between a second set of electrodes for a period of time, then repeating that cycle for an extended duration (e.g., over a period of days or weeks).

In order to generate the TTFields, current is applied to each electrode of the transducer array, enabling the transcutaneous delivery of electric current to a patient. In general, the efficacy of the TTFields depends upon several factors, including the field direction, the field frequency, the field strength, compliance, and tumor type. The requirement for a patient to wear the transducer arrays on a body part for extended periods of time could potentially impact device usage and patient compliance. Additionally, the prior art teaches electrodes made from rigid and/or inflexible materials, such as ceramics and printed circuit boards (PCBs), which do not readily contour to the patient. The failure of the transducer arrays to conform to the contours of the patient at the location of TTField could reduce the effectiveness of the treatment and create skin irritations, thereby compromising patient compliance.

Because of the inflexibility of the transducer array, new and improved array assemblies that are able to conform to the contours of the patient are desired. It is to such assemblies and methods of producing and using the same, that the present disclosure is directed.

### SUMMARY OF THE INVENTION

The problems involved with immobility and inflexibility of the transducer array are solved by a transducer array comprising: an electrode assembly, operable to supply an electrical signal having an alternating current waveform at a frequency in a range from 50 kHz to 1 MHz, comprising: a substrate; an electrically conductive layer; a movable connector extending between the substrate and the electrically conductive layer, the movable connector being constructed of an electrically conductive material and movable so as to enable movement of the electrically conductive layer relative to the substrate.

The benefits of a transducer array with an adjustable and adaptable electrically conductive layer are multifold. One significant advantage of the present transducer array having the movable connector is that the movable connector enables the electrically conductive layer to continuously adapt to the movements of a patient's skin. Skin naturally moves and stretches in the X-Y plane. The movable connector allows the electrically conductive layer to move in concert with the patient's skin, thereby enhancing the electrical connection between the electrically conductive layer and the patient's skin. In addition, the present transducer array addresses the common complication of skin irritations caused or exacerbated by ongoing TTField treatments. Because the movable connector permits the electrically conductive layer to adapt to the movement of the patient's skin rather than resisting such movement, friction occurring at the skin/device interface will be reduced, thereby lowering the prevalence of skin irritations arising during and/or after TTField treatments. Lowering the incidence of this common side-effect promotes patient compliance. Thus, the present transducer array having the movable connector addresses and overcomes two primary problems of the prior art (device usage and patient compliance) and offers opportunities for improved patient outcomes.

Further, embodiments of the present transducer array comprising the movable connector enable movement of the electrically conductive layer in the z axis as well. Consequently, the movable connector allows movement of the electrically conductive layer relative to the substrate so as to position the electrically conductive layer parallel to a patient's skin when the substrate is not parallel to the patient's skin. The present transducer array's ability to adjust the position of the electrically conductive layer in three dimensions relative to the substrate means that the transducer array not only adjusts to accommodate for anatomical differences among the patient population but also continuously adapts to the ongoing movements of a particular patient's skin during TTField treatment.

The details of one or more implementations of the subject matter described in this specification are set forth in the accompanying drawings and the description below. Other aspects, features and advantages of the subject matter will become apparent from the description, the drawings, and the claims.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is a schematic diagram of an exemplary embodiment of electrodes as applied to living tissue.

FIG. 2 is an exemplary embodiment of an electronic device configured to generate a TTField constructed in accordance with the present disclosure.

FIG. 3 is a block diagram of an exemplary embodiment of a transducer array constructed in accordance with the present disclosure.

FIG. 4A is a cross-sectional view of another exemplary embodiment of a transducer array constructed in accordance with the present disclosure.

FIG. 4B is a cross-sectional view of another exemplary embodiment of a transducer array constructed in accordance with the present disclosure.

FIG. 5 is a cross-sectional view of an exemplary embodiment of an array assembly constructed in accordance with the present disclosure.

FIG. 6 is a cross-sectional view of an exemplary embodiment of an array constructed in accordance with the present disclosure.

FIG. 7 is a process flow diagram of an exemplary embodiment of a process of using the electronic apparatus to apply a TTField to a patient.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Headings are provided for convenience only and are not to be construed to limit the invention in any manner. Embodiments illustrated under any heading or in any portion of the disclosure may be combined with embodiments illustrated under the same or any other heading or other portion of the disclosure. Any combination of the elements described herein in all possible variations thereof is encompassed by the invention unless otherwise indicated herein or otherwise clearly contradicted by context.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims. In particular, where a method claim does not specifically state in the claims or description that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

As used herein, the term “concurrent therapy” is used interchangeably with the terms “combination therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Circuitry, as used herein, may be analog and/or digital components, or one or more suitably programmed processors (e.g., microprocessors) and associated hardware and software, or hardwired logic. Also, “components” may perform one or more functions. The term “component,” may include hardware, such as a processor (e.g., microprocessor), an application specific integrated circuit (ASIC), a field programmable gate array (FPGA), a combination of hardware and software, and/or the like. The term “processor” as used herein means a single processor or multiple processors working independently or together to collectively perform a task.

As used herein, the term TTField (TTFields, or TTF(s)) refers to low intensity (e.g., 1-4 V/cm) alternating electric fields of medium frequencies (about 50 kHz-1 MHz), when applied to a conductive medium, such as a human body, via electrodes as may be used, for example, to treat tumors as described in U.S. Pat. Nos. 7,016,725, 7,089,054, 7,333,852, 7,565,205, 7,805,201, and 8,244,345 by Palti (each of which is hereby incorporated herein by reference in its entirety) and in a publication by Kirson (see Eilon D. Kirson, et al., Disruption of Cancer Cell Replication by Alternating Electric Fields, Cancer Res. 2004 64:3288-3295), which is hereby incorporated herein by reference in its entirety. TTFields have been shown to have the capability to specifically affect cancer cells and serve, among other uses, for treating cancer. TTFields therapy is an approved mono-treatment for recurrent glioblastoma (GBM), and an approved combination therapy with chemotherapy for newly diagnosed GBM patients.

As used herein, the term TT Signal is an electrical signal that, when received by electrodes applied to a conductive medium, such as a human body, causes the electrodes to generate the TTField described above. The TT Signal is often an AC electrical signal.

Turning now to the inventive concept(s), certain non-limiting embodiments thereof include a transducer array, a transducer array system, and methods of implementing the system. The transducer array comprises an electrode assembly, configured to generate and operable to supply an electrical signal having an alternating current waveform at a frequency in a range from 50 kHz to 1 MHz, and comprising a substrate, an electrically conductive layer, a movable connector extending between the substrate and the electrically conductive layer, the movable connector being constructed of an electrically conductive material and movable so as to enable movement of the electrically conductive layer relative to the substrate so as to position the electrically conductive layer parallel to a patient's skin when the substrate is not parallel to the patient's skin and allow the electrically conductive layer to move with the patient's skin in the X, Y and Z directions to provide greater comfort to the patient, and a skin adjacent layer attached to the electrically conductive layer to provide an electrical interface between the electrically conductive layer and the patient's skin. Various aspects of the present disclosure are provided in detail below.

Referring now to the drawings and in particular to FIG. 1, shown therein is an exemplary embodiment of a dividing cell 10, under the influence of external TTFields (e.g., alternating fields in the frequency range of about 100 kHz to about 300 kHz), generally indicated as lines 14, generated by a first electrode 18a having a negative charge and a second electrode 18b having a positive charge.

Further shown are microtubules 22 of the dividing cell 10, that are known to have a very strong dipole moment. This strong polarization makes the microtubules 22, as well as other polar macromolecules and especially those that have a specific orientation within the cell 10 or its surroundings, susceptible to electric fields. The microtubules 22 positive charges are located at two centrioles 26 while two sets of negative poles are at a center 30 of the dividing cell 10 and point of attachment 34 of the microtubules 22 to the cell membrane. The locations of the charges form sets of double dipoles and therefore are susceptible to electric fields of differing directions. In one embodiment, the cells go through electroporation, that is, DNA or chromosomes are introduced into the cells using a pulse of electricity to briefly open pores in the cell membranes.

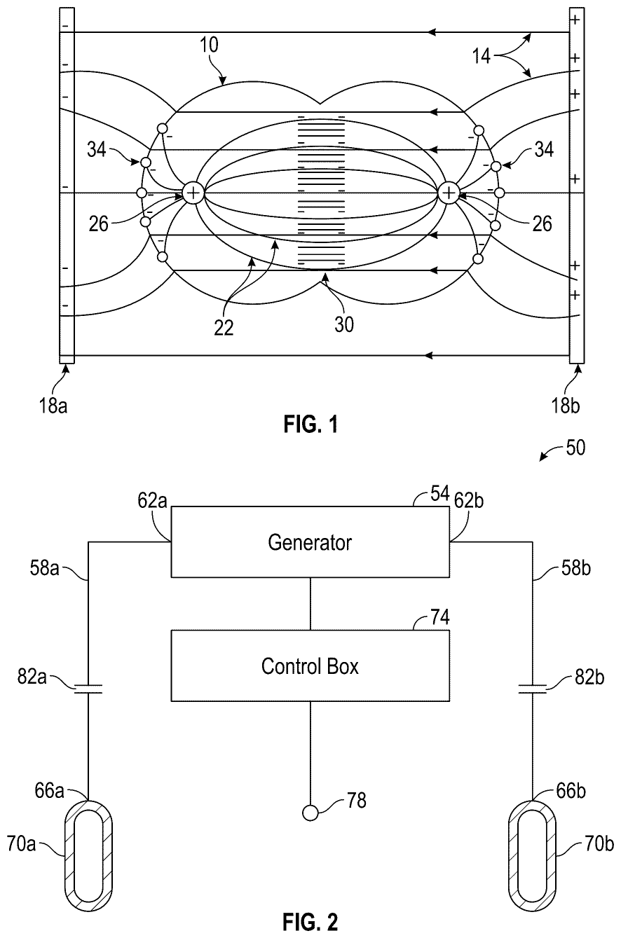

Turning now to FIG. 2, the TTFields described above that have been found to advantageously destroy tumor cells may be generated by an electronic apparatus 50. FIG. 2 is a simple schematic diagram of the electronic apparatus 50 illustrating major components thereof.

The electronic apparatus 50 includes an electric field generator 54, a pair of conductive leads 58a, 58b (including first conductive lead 58a and second conductive lead 58b, which may be referred to herein as simply conductive leads 58), a first transducer array 70a, and a second transducer array 70b. The transducer arrays may be referred to herein individually as simply transducer array 70.

The first conductive lead 58a includes a first end 62a and a second end 66a. The second conductive lead 58b includes a first end 62b and a second end 66b. The first end 62a of the first conductive lead 58a is conductively attached to the electric field generator 54 and the first end 62b of the second conductive lead 58b is conductively attached to the electric field generator 54. The electric field generator 54 generates desirable electric signals (TT signals) in the shape of waveforms or trains of pulses as an output. The second end 66a of the first conductive lead 58a is connected to the first transducer array 70a and the second end 66b of the second conductive lead 58b is connected to the second transducer array 70b. Both of the first transducer array 70a and the second transducer array 70b are activated by the electric signals (e.g., TT signals, wave forms).

The first transducer array 70a and the second transducer array 70b, being activated by the electric signals, cause an electrical current to flow between the first transducer array 70a and the second transducer array 70b. The electrical current generates an electric field (i.e., TTField), having a frequency and an amplitude, to be generated between the first transducer array 70a and the second transducer array 70b.

While the electronic apparatus 50 shown in FIG. 2 comprises only two transducer arrays 70 (the first transducer array 70a and the second transducer array 70b), in some embodiments, the electronic apparatus 50 may comprise more than two transducer arrays 70. Likewise, in some embodiments, the electronic apparatus 50 may comprise more or fewer conductive leads 58.

The electric field generator 54 generates an alternating voltage wave form at frequencies in the range from about 50 kHz to about 1 MHz (preferably from about 100 kHz to about 300 kHz) (i.e., the TTFields). The required voltages are such that an electric field intensity in tissue within the treatment area is in the range of about 0.1 V/cm to about 10 V/cm.

To achieve this field, the potential difference between two conductors, in each of the first transducer array 70a and the second transducer array 70b, is determined by the total impedance measured between the first transducer array 70a and the second transducer array 70b. The total impedance between the first transducer array 70a is determined by taking into account factors including, but not limited to, the relative impedances of the system components, e.g., a fraction of the electric field on each component is given by that component's impedance divided by a total circuit impedance, and the impedance of the patient's body which may be influenced by where the first transducer array 70a and the second transducer array 70b are located on the patient's body, tissue types between the first transducer array 70a and the second transducer array 70b, and resistance levels at the skin—array interface.

The electric field generator 54 supplies a voltage and an amperage to the first transducer array 70a and the second transducer array 70b so as to cause the first transducer array 70a and the second transducer array 70b to supply the tumor treating fields at a desired level. The amount of voltage and/or amperage may depend upon the body part to which the first transducer array 70a and the second transducer array 70b are applied.

Without exceeding a comfortability threshold discussed below, the electric field generator 54 applies a minimum amount of amperage (e.g., 0.5A, 0.7A, 0.9A, 1A, etc.) at voltages within an exemplary range from 100V to 240V. The range of amperages may be from 0.5A to 4A.

Amperages supplied by the electric field generator 54 will vary depending upon the particular design of the first transducer array 70a and the second transducer array 70b, as well as the location of the first transducer array 70a and the second transducer array 70b on the patient's body. For example, when the first transducer array 70a and the second transducer array 70b are applied to the patient's head, the maximum amperage may be 2A. When the first transducer array 70a and the second transducer array 70b are applied to the patient's torso, however, the maximum amperage may be 4A.

In certain particular (but non-limiting) embodiments, the first transducer array 70a and the second transducer array 70b generate an alternating electric current and field within a target region of a patient. The target region typically comprises at least one tumor, and the generation of the alternating electric current and field selectively destroys or inhibits growth of the tumor.

The alternating electric current and field may be generated at any frequency that selectively destroys or inhibits growth of the tumor. Generally, TTFields with a low to intermediate frequency in the range between 50 kHz and 1 MHz have been found to be effective in tumor treatment.

For example (but not by way of limitation), the alternating electric current and field may have a frequency of about 50 kHz, about 75 kHz, about 100 kHz, about 125 kHz, about 150 kHz, about 175 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 525 kHz, about 550 kHz, or about 576 kHz, as well as a range formed from any of the above values (e.g., a range of from about 50 kHz to about 576 kHz, a range of from about 100 kHz to about 150 kHz, a range of from about 150 kHz to about 300 kHz, etc.), and a range that combines two integers that fall between two of the above-referenced values (e.g., a range of from about 32 kHz to about 333 kHz, a range of from about 78 kHz to about 298 kHz, etc.).

In certain particular (but non-limiting) embodiments, the alternating electric current and field may be imposed at two or more different frequencies. When two or more frequencies are present, each frequency is selected from any of the above-referenced values, or a range formed from any of the above-referenced values, or a range that combines two integers that fall between two of the above-referenced values. As used herein, the alternating electric field may be referred to as the electric field or as the TTField.

In order to optimize the electric field (i.e., TTField) distribution, the first transducer array 70a and the second transducer array 70b may be configured differently depending upon the application in which the pair of transducer arrays 70a and 70b are to be used. The pair of transducer arrays 70a and 70b, as described herein, are externally applied to a patient, that is, are generally applied to the patient's skin, in order to apply the electric current, and electric field (TTField) thereby generating current within the patient's tissue. Generally, the pair of first and second transducer arrays 70a and 70b are placed on the patient's skin by a user such that the electric field is generated across patient tissue within a treatment area. TTFields that are applied externally can be of a local type or widely distributed type, for example, the treatment of skin tumors and treatment of lesions close to the skin surface.

In one embodiment, the user may be a medical professional, such as a doctor, nurse, therapist, or other person acting under the instruction of a doctor, nurse, or therapist. In another embodiment, the user may be the patient, that is, the patient may place the first transducer array 70a and the second transducer array 70b on their treatment area.

Optionally, and according to another exemplary embodiment, the electronic apparatus 50 includes a control box 74 and/or a temperature sensor 78 coupled to the control box 74, which are included to control the amplitude of the electric field so as not to generate excessive heating in the treatment area.

When the control box 74 is included, the control box 74 may control the output of the electric field generator 54 causing the output to remain constant at a value preset by the user. Alternatively, the control box 74 sets the output at a maximal value that does not cause excessive heating of the treatment area. In either of the above cases, the control box 74 may issue a warning, or the like, when a temperature of the treatment area (as sensed by temperature sensor 78) exceeds a preset limit.

The temperature sensor 78 may be mechanically connected to and/or otherwise associated with the first transducer array 70a or the second transducer array 70b so as to sense the temperature of the treatment area at either one or both of the first transducer array 70a or the second transducer array 70b.

In one embodiment, the control box 74 may turn off, or decrease power of the TT Signal generated by the electrical field generator 54, if a temperature sensed by the temperature sensor 78 meets or exceeds a comfortability threshold. In one embodiment, the comfortability threshold is the temperature at which a patient would be made uncomfortable while using the first transducer array 70a and the second transducer array 70b. In one embodiment, the comfortability threshold is a temperature at or about 40 degrees Celsius. In one embodiment, the comfortability threshold is a temperature of between about 39 degrees Celsius and 42 degrees Celsius, or a specific selected temperature between about 39 degrees Celsius and 42 degrees Celsius.

The conductive leads 58 may be standard isolated conductors with a flexible metal shield, preferably grounded thereby preventing spread of any electric field generated by the conductive leads 58.

The first transducer array 70a and the second transducer array 70b may have specific shapes and positioning so as to generate the TTField of a desired configuration, direction, and intensity at the treatment area and only at that treatment area so as to focus the treatment.

The specifications of the electronic apparatus 50 as a whole and its individual components are largely influenced by the fact that at the frequency of the TTFields (50 kHz-1 MHz), living systems behave according to their “Ohmic”, rather than their dielectric properties.

In one embodiment, as an optional feature and to protect the patient from any current due to DC voltage or DC offset voltage passing through the patient, the conductive leads 58 may include a DC blocking component, such as a blocking capacitor 82 (shown in FIG. 2 as a first blocking capacitor 82a and a second blocking capacitor 82b), to block DC current from passing to the first transducer array 70a and second transducer array 70b. The location of the blocking capacitor 82 may vary. For example, the blocking capacitors 82a and 82b may be proximate to the first end 62a (or 62b) of the lead 58a (or 58b) and the electric field generator 54, proximate to the second end 66a (or 66b) of the lead 58a (or 58b) and one of the transducer arrays 70a (or 70b), and/or an element of one of the transducer arrays 70a (or 70b).

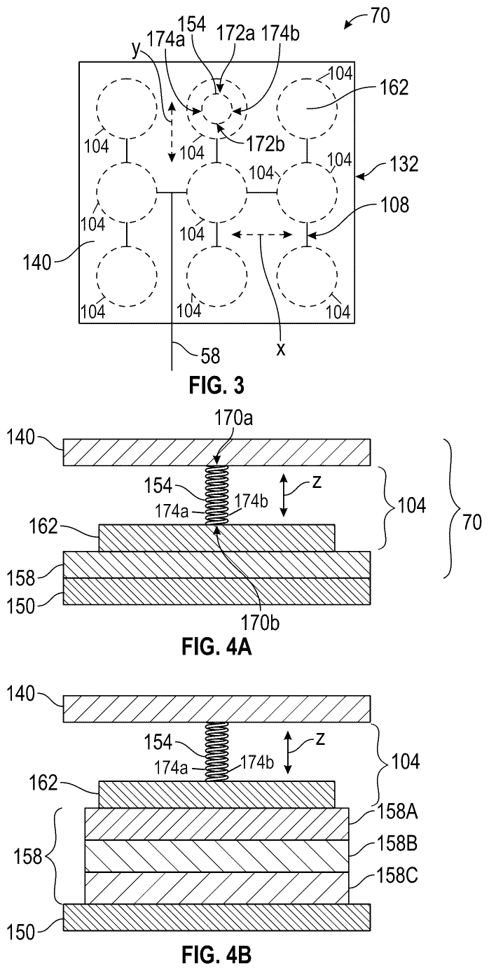

Referring now to FIG. 3, shown therein is a diagram of an exemplary embodiment of the transducer array 70 constructed in accordance with the present disclosure. In the example shown, the transducer array 70 is provided with a rectangular shape, or substantially rectangular shape with an outer edge 132. However, it should be understood that the transducer array 70 can be provided with any type of shape such as a polygon, circle, or fanciful shape. Further, the transducer array 70 may be constructed such as to be cut and/or shaped at a point of use so as to be custom fitted for a particular part of a particular patient.

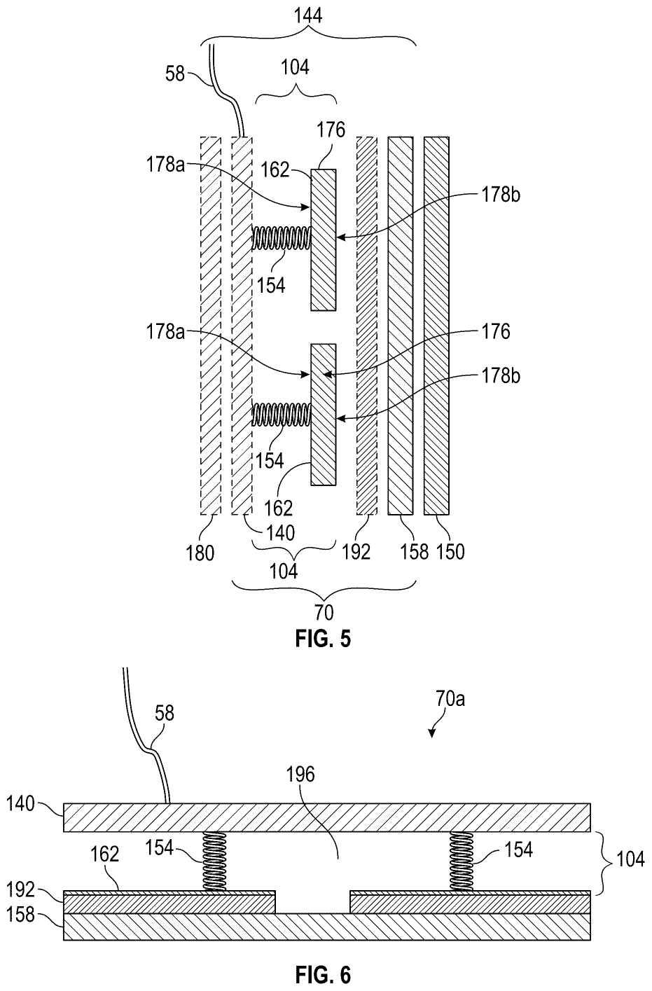

The transducer array 70 may include a conductive skin adjacent layer 158 (FIGS. 4A-5), a substrate 140, and one or more electrode assembly 104 (which may be referred to herein as electrode assembly 104).

The skin adjacent layer 158 may be any of various commercially available conductive gels or conductive adhesives such as those comprising dry carbon, as illustrated by the embodiment shown in FIG. 4B and described in more detail below.

The substrate 140 may be a printed circuit board, such as a flexible printed circuit board having conductive traces. The substrate 140 may supply the TT signals to a movable connector 154 of the electrode assembly 104, such as through the conductive traces.

As shown in the example illustrated in FIG. 3, the transducer array 70 may comprise multiple electrode assemblies 104. In one embodiment, the electrode assembly 104 may be, for example, about 2 cm in diameter. The electrode assemblies 104 may be interconnected to one another via one or more flex wires 108. The electrode assemblies 104 may be each connected to the substrate 140. The electrode assembly 104 is operable to supply an electrical signal having an alternating current waveform at a frequency in a range from 50 kHz to 1 MHz.

Referring to FIG. 4A, each electrode assembly 104 may include an electrically conductive layer 162 and the movable connector 154. The moveable connector 154 may extend between the electrically conductive layer 162 and the substrate 140. The substrate 140 may oppose and be spaced a distance from the electrically conductive layer 162. The movable connector 154 is constructed of an electrically conductive material. The electrically conductive material may be, for example, aluminum, stainless steel, or copper. Because the movable connector 154 is constructed of an electrically conductive material, the movable connector 154 is configured to supply the TT signals to the electrically conductive layer 162 via the substrate 140 from the generator 54.

The movable connector 154 is configured to be movable so as to enable movement of the electrically conductive layer 162 relative to the substrate 140, so as to position the electrically conductive layer 162 parallel to a patient's skin 150 when the substrate 140 is not parallel to the patient's skin 150. The movable connector 154 is configured to allow the electrically conductive layer 162 and the skin adjacent layer 158 to move with the patient's skin 150, thereby causing less friction and irritation to the patient's skin and improving the patient's comfort.

In an exemplary embodiment, the movable connector 154 has a first end 170a, a second end 170b, a front 172a, a back 172b, a first side 174a, and a second side 174b. The movable connector 154 has a first axis (shown as the Z axis in FIG. 4A) extending between the first end 170a and the second end 170b, a second axis (shown as the y axis in FIG. 3) extending between the front 172a and the back 172b, and a third axis (shown as the x axis in FIG. 3) extending between the first side 174a and the second side 174b.

The first end 170a of the movable connector 154 is electrically connected to the substrate 140 to receive the TT signals from the substrate 140. The second end 170b of the movable connector 154 is electrically connected to the electrically conductive layer 162 such that TT signals pass through the movable connector 154 and are supplied to the electrically conductive layer 162.

In some embodiments, the movable connector 154 may have an initial state with a memory, such that the movable connector 154 is structured to permit the electrically conductive layer 162 to move in three dimensions (e.g., the X, Y, and Z dimensions) and then the movable connector 154 provides a bias to move the electrically conductive layer 162 to the initial state.

In other embodiments, for example, if the moveable connector 155 is a ball joint, the movable connector 154 does not have a memory but permits the electrically conductive layer 162 to move in at least two or three dimensions.

The movable connector 154 may permit movement of the first end 170a of the movable connector 154 relative to the second end 170b of the movable connector 154 along the first axis, the second axis, and/or the third axis.

In FIG. 3, the direction of movement of the first end 170a relative to the second end 170b of the movable connector 154, i.e., the electrically conductive layer 162 relative to the substrate 140 is shown in the directions along the x axis and the y axis. Additionally, in FIG. 4, the direction of movement of the first end 170a relative to the second end 170b of the movable connector 154, i.e., the electrically conductive layer 162 relative to the substrate 140 is shown as movable vertically along the z axis.

An example of a suitable movable connector 154 is a conductive ball joint having the first end 170a, the second end 170b, and an intermediate ball joint between the first end 170a and the second end 170b. An example of a suitable movable connector 154 is a conductive ball bearing element. Another example of a suitable movable connector 154 as shown in FIG. 4A, FIG. 4B, FIG. 5, and FIG. 6 is a micro spring comprising metal or other conductive material. By way of illustration but not limitation, micro springs useful for one embodiment of this disclosure include micro springs having an inner diameter in size from 0.36 mm to 115 mm. Another embodiment comprises conductive micro springs having an inner spring diameter in the range of about 0.1 mm to 3.2 mm. In another embodiment of the present disclosure, the movable connector 154 is a spring contact such as those commercially available by Alps Alpine, which has its headquarters at 1-7 Yukigayaotsukamachi, Ota-ku, Tokyo 145-8501. An example of a suitable spring contact useful as a movable connector 154 in an embodiment of the present disclosure is the Alps Alpine SCTA Spring Contact Micro Clips, having a height of approximately 1.2 mm.

In some embodiments, the electrically conductive layer 162 of the transducer array 70 may comprise a plurality of separate electrode elements 176. In this particular embodiment, each of the spatially disposed electrode elements 176 has a first surface 178a and a second surface 178b opposite the first surface 178a, the first surface 178a in contact with the movable connector 154. The spatially disposed electrode elements 176 are adjacently disposed and may be provided in a common plane. Although only two of the movable connectors 154 and the spatially disposed electrode elements 176 are shown in FIG. 5, it should be understood that the transducer array 70 could include more than two of the movable connectors 154 and the spatially disposed electrode elements 176. For example, in some embodiments the transducer array may include 8, 9, or 10 of the movable connectors 154 and the spatially disposed electrode elements 176.

In some embodiments, the transducer array 70 may optionally comprise a dielectric layer 192 positioned adjacent to the second surface 178b of the electrically conductive layer 162. The inventive transducer array 70 may also include ceramic elements that are disc-shaped, ceramic elements that are not disc-shaped, and non-ceramic dielectric materials positioned between the electrically conductive layer 162 and the skin adjacent layer 158 of the transducer array 70 over a plurality of spatially disposed electrode elements 176 in the electrically conductive layer 162 (see FIG. 6). Examples of non-ceramic dielectric materials positioned over a plurality of electrode elements 176 include polymer films disposed over arrays on a printed circuit board or over flat pieces of metal. Other alternative constructions for implementing the transducer array 70 may also be used, as long as they are capable of delivering TTFields to the body of the patient.

Referring now to FIG. 5, shown therein is a cross section of an exemplary embodiment of a transducer array assembly 144 constructed in accordance with the present disclosure. The transducer array assembly 144 generally comprises an electrode assembly 104 as described above, comprising the substrate 140, the electrically conductive layer 162, the conductive movable connector 154, the skin adjacent layer 158 attached to the electrically conductive layer 162. The transducer array assembly 144 may further comprises a compression layer 180. The compression layer 180 can be made of an elastic material coated with a pressure sensitive adhesive. For example, the compression layer 180 may be a bandage. In some embodiments, the transducer array assembly 144 includes a dielectric layer 192 disposed between the electrically conductive layer 162 and the conductive skin adjacent layer 158.

The conductive skin adjacent layer 158 may be in any form that allows the composition to function in accordance with the present disclosure. For example (but not by way of limitation), the conductive skin adjacent layer 158 may be in the form of a hydrogel or a hydrocolloid. In addition, the conductive skin adjacent layer 158 may include adhesive properties. In one embodiment, the conductive skin adjacent layer 158 comprises one or more layers of a dry conductive carbon adhesive material, for example, the commercially available Omni-Wave™ material manufactured by Flexcon Company, Inc., which has its corporate headquarters at 1 Flexcon Industrial Park, Spencer, MA 01562-2642.

In some exemplary embodiments of the skin adjacent layer 158 shown in FIG. 4B, the conductive skin adjacent layer 158 may have a plurality of layers. For example, the conductive skin adjacent layer 158 may comprise three layers: a first conductive adhesive layer 158A, a flexible sheet of conductive material 158B, and a second conductive adhesive layer 158C, each of which comprise a top surface and a bottom surface. The electrically conductive layer 162 is attached to the top surface of the first conductive adhesive layer 158A. The bottom surface of the first conductive adhesive layer 158A is positioned adjacent to and attached to the top surface of the flexible sheet of conductive material 158B. The flexible sheet of conductive material 158B is maintained in electrical contact with the electrically conductive layer 162 by the first conductive adhesive layer 158A. The bottom surface of the flexible sheet of conductive material 158B is, in turn, connected to the top surface of the second adhesive layer 158C. The bottom surface of the second adhesive layer 158C is positionable adjacent to and configured to be attachable to the patient's skin 150.

Examples of suitable materials for the first conductive adhesive layer 158A and the second conductive adhesive layer 158C include, but are not limited to, the OMNI-WAVE™ adhesive compositions manufactured and sold by FLEXCON® (Spencer, MA, USA), such as the developmental product FLX068983—FLEXcon® OMNI-WAVE™ TT 200 BLACK H-502 150 POLY H-9 44PP-8; and the adhesives from ADHESIVE RESEARCH, such as ARcare® 8006 electrically conductive adhesive composition manufactured and sold by Adhesives Research, Inc. (Glen Rock, PA, USA). Alternatively, Electrically Conductive Adhesive Transfer Tape 9712 or Electrically Conductive Adhesive Transfer Tape 9713 (both manufactured by 3M) may also be used.

In some embodiments, the first conductive adhesive layer 158A and the second conductive adhesive layer 158C may comprise a conductive gel (e.g., conductive hydrogel) instead of the layer of conductive adhesive. In these embodiments, the level of adhesion between the second conductive adhesive layer 158C and the patient's skin 150 may be relatively weak and will be based on the tackiness of the conductive gel.

Examples of suitable materials for the flexible sheet of conductive material 158B include, but are not limited to, synthetic graphite, pyrolytic graphite (including, but not limited to, Pyrolytic Graphite Sheet (PGS), available from Panasonic Industry, Kadoma, Osaka, Japan), graphitized polymer film (e.g., graphitized polyimide film, including, but not limited to, that supplied by Kaneka Corp., Moka, Tochigi, Japan), or graphite foil made from compressed high purity exfoliated mineral graphite (including, but not limited to, that supplied by MinGraph® 2010A Flexible Graphite, available from Mineral Seal Corp., Tucson, Arizona, USA). In alternative embodiments, instead of using a layer of graphite, a layer of another anisotropic material or another flexible conductive material may be used.

As shown in FIG. 5, in some embodiments, the transducer array assembly 144 may include the compression layer 180. The compression layer 180 may be an exterior covering operable to cause a compression between the transducer array assembly 144 and the patient's skin 150 when the transducer array assembly 144 is placed on the patient to bias the skin adjacent layer 158 against the patient's skin 150. In an exemplary embodiment, the compression layer 180 resembles or may be an adhesive bandage. In another embodiment, the compression layer 180 is a form of clothing, for example, a shirt, an undergarment, or a pants. In one embodiment, the compression layer 170 is non-conductive so as to not interfere with the TTfield being generated by the electrically conductive layer 162.

In some embodiments, the dielectric layer 192 is provided within the transducer array 70, or the transducer array assembly 144. The dielectric layer 192 may be constructed of one or more dielectric material and configured to function as an insulator.

In some embodiments, the dielectric layer 192 includes a ceramic material. In some embodiments, the dielectric layer 192 is a flexible polymer. In some embodiments, the dielectric layer 192 comprises poly(vinylidene fluoride-trifluoroethylene-chlorotrifluoroethylene) and/or poly(vinylidene fluoride-trifluoroethylene-1-chlorofluoroethylene). Those two polymers are abbreviated herein as “Poly(VDF-TrFE-CtFE)” and “Poly(VDF-TrFE-CFE)”, respectively. These embodiments are particularly advantageous because the dielectric constant of these materials is on the order of 40.

Because the TTFields are coupled through the dielectric layer 192, and because capacitance is inversely proportional to the thickness of the dielectric layer 192, the dielectric layer 192 is preferably as thin as possible (e.g., less than 10 μm or less than 5 μm). On the other hand, the dielectric layer 192 should not be too thin because that could impair manufacturability, compromise the layer's structural integrity, and risk dielectric breakdown when the AC signals are applied. In some preferred embodiments, the dielectric layer 192 has a thickness that is at least 1 μm. In some preferred embodiments the dielectric layer 192 is between 1-3 μm thick (e.g., about 2 μm), which provides a good balance between the parameters noted above. Preferably, the thickness of the dielectric layer 192 is uniform. But in alternative embodiments, the thickness could be non-uniform.

Optionally, ceramic nanoparticles may be mixed into the Poly(VDF-TrFE-CtFE) and/or Poly(VDF-TrFE-CFE). Optionally, these ceramic nanoparticles may comprise at least one of barium titanate and barium strontium titanate.

In some embodiments, instead of forming the dielectric layer 192 from Poly(VDF-TrFE-CtFE) and/or Poly(VDF-TrFE-CFE), a different polymer that provides a high dielectric constant, and/or a high level of capacitance may be used. The requirements for these different polymers are as follows: (1) at least one frequency between 50 kHz and 576 kHz, the polymer layer has a dielectric constant of at least 20; (2) the dielectric layer 192 has a thickness of less than 20 microns; and (3) the thickness of the dielectric layer 192 multiplied by its dielectric strength is at least 200 V. Example of polymers that may be used in place of Poly(VDF-TrFE-CtFE) and/or Poly(VDF-TrFE-CFE) include the following: (1) ceramic nanoparticles mixed into at least one of Poly(VDF-TrFE), P(VDF-HFP), PVDF; and (2) barium titanate and/or barium strontium titanate ceramic nanoparticles mixed into at least one of Poly(VDF-TrFE), P(VDF-HFP), PVDF (where Poly(VDF-TrFE), P(VDF-HFP), and PVDF are, respectively poly(vinylidene fluoride-trifluoroethylene), poly(vinylidene fluoride-hexafluoropropylene) and poly(vinylidene fluoride).).

In some embodiments, the thickness of the dielectric layer 192 is less than 10 μm, and in some preferred embodiments, the thickness of the dielectric layer 192 is less than 5 μm. In some preferred embodiments, the thickness of the dielectric layer 192 multiplied by its dielectric strength is at least 476 V. In other embodiments, the dielectric layer 192 has a dielectric constant of at least 20 measured at 200 kHz.

Referring now to FIG. 6, shown therein is a cross-sectional view of an exemplary embodiment of the transducer array 70a constructed in accordance with the present disclosure. As shown in FIG. 6, the transducer array 70a comprises at least one electrode assembly 104 comprising the substrate 140, the electrically conductive layer 162, the movable connector 154, the conductive skin adjacent layer 158, and the dielectric layer 192 positioned between the electrically conductive layer 162 and the conductive skin adjacent layer 158. In one embodiment, the electrically conductive layer 162, the moveable connectors 154, the substrate 140, and the conductive skin adjacent layer 158 (and, optionally, the dielectric layer 192) of the transducer array 70a cooperate to define at least one opening 196 between the movable connectors 154 and disposed at least partially through the transducer array 70a.

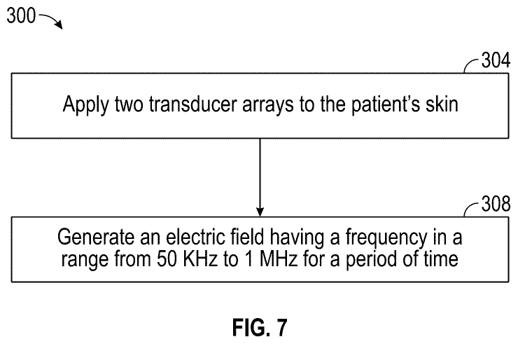

Referring now to FIG. 7, shown therein is an exemplary embodiment of a process 300 of using the electronic apparatus 50 to apply a TTField to a patient. The process 300 generally comprises the steps of: applying two transducer arrays 70, 70a (and/or transducer array assembly 144) to the patient's skin (step 304) and generating an alternating electric field (TTField) having a frequency in a range of from about 50 kHz to about 1 MHz for a period of time (step 308).

In one embodiment, the step of applying two transducer arrays 70, 70a (and/or transducer array assembly 144) to the patient's skin (step 304) includes applying two or more transducer arrays 70, 70a (and/or transducer array assembly 144) to the patient's skin.

A method of the present invention comprises applying at least two transducer arrays 70, 70a (and/or transducer array assembly 144) defining conductive regions to a skin of a patient. The conductive regions defined by the transducer arrays 70, 70a (and/or transducer array assembly 144) are coupled to the electric field generator 54 before or after applying the at least two conductive regions to patient's skin, the electric field generator 54 being configured to generate an electrical signal having an alternating current waveform at a frequency in a range from 50 kHz to 1 MHz.

Each of these conductive regions has an electrode assembly 104 comprising the electrically conductive layer 162 and the electrically conductive movable connector 154 extending between the electrically conductive layer 162 and the substrate 140, and the conductive skin adjacent layer 158 attached to the electrically conductive layer 162. The movable connector 154 is operable to supply the electrical signal to the electrically conductive layer 162 and to maintain the electrically conductive layer 162 parallel to the patient's skin 150 while allowing the electrically conductive layer 162 to move with the patient's skin, the electrically conductive layer 162 is electrically coupled with a patient's skin 150. The electric field generator 54 is activated to supply the electrical signal to the conductive regions, thereby supplying electrical current to the patient's skin 150 through the conductive skin adjacent layer 158.

In some embodiments, the number of transducer arrays 70, 70a (and/or transducer array assembly 144) applied to the patient's skin 150 is determined by a number of transducer arrays 70 needed to apply a TTField having a therapeutic benefit as determined by the user, such as by a medical professional.

The step of applying two transducer arrays (step 304) may be performed by the user. In one embodiment, before applying the selected transducer arrays to the patient's skin, the patient's skin may need to be cleaned (e.g., such as but not limited to, cleansing of the skin of foreign matter or biological matter and shaving of the skin, if necessary).

The step of generating an alternating electric field (TTField) (step 308) may be performed by the electric field generator 54. In one embodiment, step 308 may be performed more than one time and the period of time for which the step 308 is performed a first time may be the same as or different from the period of time for which the step 308 is performed a second time (or other period(s) of time beyond the second time).

In some embodiments, step 308 is only performed once before the process 300 is repeated. There may be a time period between each time the process 300 is repeated. Each time the process 300 is repeated, the time period may be the same as or different from the previous time period. Each time the process 300 is repeated, the selected transducer array or plurality of transducer arrays may be placed in the same or a different position on the patient's skin.

In one embodiment, prior to generating an alternating electric field (TTField) having a frequency in a range of from about 50 kHz to about 1 MHz for a period of time (step 308), the user connects, or electrically couples, the selected transducer array 70 to the electric field generator 54.

### Illustrative Embodiments

Clause 1. A transducer array, comprising: an electrode assembly, operable to supply an electrical signal having an alternating current waveform at a frequency in a range from 50 kHz to 1 MHz, comprising: a substrate; an electrically conductive layer; a movable connector extending between the substrate and the electrically conductive layer, the movable connector being constructed of an electrically conductive material and movable so as to enable movement of the electrically conductive layer relative to the substrate so as to position the electrically conductive layer parallel to a patient's skin when the substrate is not parallel to the patient's skin; and a conductive skin adjacent layer attached to the electrically conductive layer, to provide an electrical interface between the electrically conductive layer and the patient's skin.
Clause 2. The transducer array of Clause 1, wherein the substrate is a printed circuit board.
Clause 3. The transducer array of Clause 1 or 2, wherein the movable connector comprises at least one of a conductive ball bearing element and a conductive ball joint.
Clause 4. The transducer array of any one of Clauses 1-4, wherein the movable connector has a first end, a second end, a front, a back, a first side and a second side; the movable connector having a first axis extending between the first end and the second end, a second axis extending between the front and the back, and a third axis extending between the first side and the second side; the movable connector having an initial state with a memory, and being movable in at least two of the first axis, the second axis and the third axis.
Clause 5. The transducer array of any one of Clauses 1, 2, and 4, wherein the movable connector is a micro spring.
Clause 6. The transducer array of Clause 5, wherein the micro spring has an inner spring diameter in the range of about 0.1 mm to 3.2 mm.
Clause 7. The transducer array of any one of Clauses 1, 2, and 4, wherein the movable connector is a spring contact.
Clause 8. The transducer array of Clause 7, wherein the spring contact has a height of about 1.0 mm-2.0 mm.
Clause 9. The transducer array of any one of Clauses 1-8,wherein the electrically conductive layer comprises a plurality of spatially disposed electrode elements.
Clause 10. The transducer array of Clause 9, wherein each of the spatially disposed electrode elements has a first surface and a second surface opposite of the first surface, the first surface in contact with the movable connector, and wherein the transducer array further comprises a dielectric layer, positioned adjacent to the second surface of the electrically conductive layer.
Clause 11. The transducer array of Clause 10, wherein the dielectric layer comprises a plurality of spatially disposed dielectric elements positioned adjacent to the second surface of respective electrode elements.
Clause 12. The transducer array of Clause 11, wherein the dielectric elements include a flexible polymer material.
Clause 13. The transducer array of Clause 11, wherein the dielectric elements include a ceramic material.
Clause 14. The transducer array of any one of Clauses 1-13, wherein the conductive skin adjacent layer comprises conductive gel.
Clause 15. The transducer array of any one of Clauses 1-14, wherein the conductive skin adjacent layer comprises a conductive adhesive.
Clause 16. The transducer array of any one of Clauses 1-15, wherein the electrode assembly further comprises a compression layer, operable to apply a force upon the movable connector, causing the movable connector to move relative to the substrate when the transducer array is placed upon the skin of a patient.
Clause 17. A system for delivering TTFields to a body of a subject, the system comprising: an electric field generator configured to generate an electrical signal having an alternating current waveform at a frequency in a range from 50 kHz to 1 MHz; a first conductive lead electrically coupled to the electric field generator, the first conductive lead configured to carry the electrical signal; a first transducer array coupled to the first conductive lead, the first transducer array having a first electrode assembly comprising a first substrate, a first electrically conductive layer, a first movable connector extending between the first substrate and the first electrically conductive layer, and electrically connected to the first conductive lead, and a first conductive skin adjacent layer, the first movable connector constructed of a conductive material operable to carry the electrical signal to the first electrically conductive layer and to maintain the first electrically conductive layer parallel to a patient's skin when the first transducer array is applied to the patient's skin; a second conductive lead electrically coupled to the electric field generator, the second conductive lead configured to carry the electrical signal; and a second transducer array coupled to the second conductive lead, the second transducer array receiving the electrical signal from the second conductive lead, the second transducer array comprising a second electrode assembly connected to a second conductive skin adjacent layer.
Clause 18. The system of Clause 17, wherein the first substrate is a printed circuit board.
Clause 19. The system of Clause 17 or 18, wherein the first movable connector is movable so as to enable movement of the electrically conductive layer relative to the first substrate so as to position the first electrically conductive layer parallel to a patient's skin when the first substrate is not parallel to the patient's skin.
Clause 20. The system of any one of Clauses 17-19, wherein the first movable connector comprises a conductive ball bearing element.
Clause 21. The system of any one of Clauses 17-20, wherein the first movable connector has a first end, a second end, a front, a back, a first side and a second side; the first movable connector having a first axis extending between the first end and the second end, a second axis extending between the front and the back, and a third axis extending between the first side and the second side; the first movable connector having an initial state with a memory, and being movable in at least two of the first axis, the second axis and the third axis.
Clause 22. The system of any one of Clauses 17-19 and 21,wherein the first movable connector is a micro spring.
Clause 23. The system of Clause 22, wherein the micro spring has an inner spring diameter in the range of about 0.1 mm to 3.2 mm.
Clause 24. The system of one of Clauses 17-19 and 21, wherein the first movable connector is a spring contact.
Clause 25. The system of Clause 24, wherein the spring contact has a height of about 1.0 mm-2.0 mm.
Clause 26. The system of any one of Clauses 17-25, wherein the first electrically conductive layer comprises a plurality of spatially disposed electrode elements.
Clause 27. The system of Clause 26, wherein each of the spatially disposed electrode elements has a first surface and a second surface opposite of the first surface, the first surface in contact with the first movable connector, and wherein the first transducer array further comprises a first dielectric layer positioned adjacent to the second surface of electrode elements.
Clause 28. The system of Clause 27, wherein the first dielectric layer comprises a plurality of spatially disposed dielectric elements positioned adjacent to the second surface of respective electrode elements.
Clause 29. The system of Clause 28, wherein the spatially disposed dielectric elements include a flexible polymer material.
Clause 30. The system of Clause 28, wherein the dielectric elements include a ceramic material.
Clause 31. The system of any one of Clauses 17-30, wherein the first and second conductive skin adjacent layers comprise conductive gel.
Clause 32. The system of any one of Clauses 17-31, wherein the first and second conductive skin adjacent layers comprise a conductive adhesive.
Clause 33. The system of any one of Clauses 17-32, wherein the first electrode assembly further comprises a compression layer, operable to apply a force upon the first movable connector, causing the first movable connector to move relative to the first substrate when the first transducer array is placed upon a patient's skin.
Clause 34. A method, comprising: applying at least two conductive regions to a skin of a patient, coupling the conductive regions to an electric field generator before or after applying the at least two conductive regions to a skin of the patient, the electric field generator configured to generate an electrical signal having an alternating current waveform at a frequency in a range from 50 kHz to 1 MHz, each conductive region having an electrode assembly comprising a substrate, an electrically conductive layer, an electrically conductive movable connector extending between the substrate and the electrically conductive layer, and a conductive skin adjacent layer attached to the electrically conductive layer, the movable connector being operable to supply the electrical signal to the electrically conductive layer and to maintain the electrically conductive layer parallel to the skin, the electrically conductive layer being electrically coupled with a patient's skin; and activating the electric field generator to supply the electrical signal to the conductive regions, thereby supplying electrical current to the patient through the conductive skin adjacent layer.
Clause 35. The method of Clause 34, wherein the substrate is a printed circuit board and wherein coupling the conductive regions to the electric field generator including passing the alternating current waveform through at least one trace on the printed circuit board.
Clause 36. The method of Clause 34 or 35, wherein the movable connector is movable so as to enable movement of the electrically conductive layer relative to the substrate so as to position the electrically conductive layer parallel to a patient's skin when the substrate is not parallel to the patient's skin, and wherein the step of applying the at least two conductive regions to the skin of the patient includes applying the conductive regions so that the electrically conductive layer is parallel to a patient's skin and the substrate is not parallel to the patient's skin.
Clause 37. The method of Clause 36, wherein the movable connector comprises at least one of a conductive ball bearing element and a conductive ball joint, and wherein the step of applying the at least two conductive regions to the skin of the patient includes moving the conductive ball bearing element or the conductive ball joint to position the electrically conductive layer parallel to the skin.
Clause 38. The method of any one of Clauses 34-37, wherein the movable connector has a first end, a second end, a front, a back, a first side and a second side; the movable connector having a first axis extending between the first end and the second end, a second axis extending between the front and the back, and a third axis extending between the first side and the second side; the movable connector having an initial state with a memory, and being movable in the first axis, the second axis and the third axis; and wherein the step of applying the at least two conductive regions to the skin of the patient includes moving the movable connector in at least two of the first axis, the second axis and the third axis.
Clause 39. The method of any one of Clauses 34-36 and 38, wherein the movable connector is a micro spring having a first end with a first orientation and a second end with a second orientation, and wherein the step of applying the at least two conductive regions to the skin of the patient includes moving the first end relative to the second end to thereby change the second orientation relative to the first orientation.
Clause 40. The method of Clause 39, wherein the micro spring has an inner spring diameter in the range of about 0.1 mm to 3.2 mm.
Clause 41. The method of any one of Clauses 34-36 and 38, wherein the movable connector is a spring contact having a first end with a first orientation and a second end with a second orientation, and wherein the step of applying the at least two conductive regions to the skin of the patient includes moving the first end relative to the second end to thereby change the second orientation relative to the first orientation.
Clause 42. The method of Clause 41, wherein the spring contact has a height of about 1.0 mm-2.0 mm.
Clause 43. The method of any one of Clauses 34-42, wherein each of the conductive regions includes a transducer array of any one of Clauses 17-32.
Clause 44. The method of any one of Clauses 34-43, wherein the electrode assembly of each conductive region further comprises a compression layer, operable to apply a force upon the movable connector, and wherein the step of applying the at least two conductive regions to the skin of the patient includes applying force by the compression layer upon the movable connector, thereby causing the movable connector to move relative to the substrate.
Clause 45. A transducer array, comprising: an electrode assembly, operable to supply an electrical signal having an alternating current waveform at a frequency in a range from 50 kHz to 1 MHz, comprising: a substrate; an electrically conductive layer; a movable connector extending between the substrate and the electrically conductive layer, the movable connector being constructed of an electrically conductive material and movable so as to enable movement of the electrically conductive layer relative to the substrate so as to permit movement of the electrically conductive layer responsive to movement of the patient's skin; and a conductive skin adjacent layer attached to the electrically conductive layer, to provide an electrical interface between the electrically conductive layer and the patient's skin.
Clause 46. The transducer array of Clause 45, wherein the substrate is a printed circuit board.
Clause 47. The transducer array of Clause 45 or 46, wherein the movable connector comprises at least one of a conductive ball joint element, and a conductive ball bearing.
Clause 48. The transducer array of any one of Clauses 45-47, wherein the movable connector has a first end, a second end, a front, a back, a first side and a second side; the movable connector having a first axis extending between the first end and the second end, a second axis extending between the front and the back, and a third axis extending between the first side and the second side; the movable connector having an initial state with a memory, and being movable in at least two of the first axis, the second axis and the third axis.
Clause 49. The transducer array of any one of Clauses 45, 46, and 48, wherein the movable connector is a micro spring.
Clause 50. The transducer array of Clause 49, wherein the micro spring has an inner spring diameter in the range of about 0.1 mm to 3.2 mm.
Clause 51. The transducer array of any one of Clauses 45, 46, and 48, wherein the movable connector is a spring contact.
Clause 52. The transducer array of Clause 51, wherein the spring contact has a height of about 1.0 mm-2.0 mm.
Clause 53. The transducer array of any one of Clauses 45-52, wherein the electrically conductive layer comprises a plurality of spatially disposed electrode elements.
Clause 54. The transducer array of Clause 53, wherein each of the spatially disposed electrode elements has a first surface and a second surface opposite of the first surface, the first surface in contact with the movable connector, and wherein the transducer array further comprises a dielectric layer, positioned adjacent to the second surface of the electrically conductive layer.
Clause 55. The transducer array of claim 54, wherein the dielectric layer comprises a plurality of spatially disposed dielectric elements positioned adjacent to the second surface of respective electrode elements.
Clause 56. The transducer array of Clause 55, wherein the dielectric elements include a flexible polymer material.
Clause 57. The transducer array of Clause 55, wherein the dielectric elements include a ceramic material.
Clause 58. The transducer array of any one of Clauses 45-57, wherein the conductive skin adjacent layer comprises conductive gel.
Clause 59. The transducer array of any one of Clauses 45-58, wherein the conductive skin adjacent layer comprises a conductive adhesive.
Clause 60. The transducer array of any one of Clauses 45-59, wherein the electrode assembly further comprises a compression layer, operable to apply a force upon the movable connector, causing the movable connector to move relative to the substrate when the transducer array is placed upon the skin of a patient.

From the above description, it is clear that the inventive concepts disclosed and claimed herein are well adapted to carry out the objects and to attain the advantages mentioned herein, as well as those inherent in the invention. While exemplary embodiments of the inventive concepts have been described for purposes of this disclosure, it will be understood that numerous changes may be made which will readily suggest themselves to those skilled in the art and which are accomplished within the spirit of the inventive concepts disclosed and claimed herein.

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features and steps are recited in the claims and/or disclosed in the specification, these combinations are not intended to limit the disclosure. In fact, many of these features and steps may be combined in ways not specifically recited in the claims and/or disclosed in the specification. Although each dependent claim listed below may directly depend on only one other claim, the disclosure includes each dependent claim in combination with every other claim in the claim set.

Similarly, although each illustrative embodiment listed above may directly depend on only one other illustrative embodiment, the disclosure includes each illustrative embodiment in combination with every other illustrative embodiment in the set of illustrative embodiments for each mode of the inventive concepts disclosed herein.

No element, act, or instruction used in the present application should be construed as critical or essential to the invention unless explicitly described as such outside of the preferred embodiment. Further, the phrase “based on” is intended to mean “based, at least in part, on” unless explicitly stated otherwise.

What is claimed is:

1. A transducer array, comprising:
an electrode assembly, operable to supply an electrical signal having an alternating current waveform at a frequency in a range from 50 kHz to 1 MHz, comprising:
a substrate;
an electrically conductive layer;
a movable connector extending between the substrate and the electrically conductive layer, the movable connector being constructed of an electrically conductive material and movable so as to enable movement of the electrically conductive layer relative to the substrate so as to position the electrically conductive layer parallel to a patient's skin when the substrate is not parallel to the patient's skin when the transducer array is applied to a patient's skin; and
a conductive skin adjacent layer attached to the electrically conductive layer, configured to provide an electrical interface between the electrically conductive layer and the patient's skin when the transducer array is applied to a patient's skin.

2. The transducer array of claim 1, wherein the substrate is a printed circuit board.

3. The transducer array of claim 1, wherein the movable connector comprises at least one of a conductive ball bearing element and a conductive ball joint.

4. The transducer array of claim 1, wherein the movable connector has a first end, a second end, a front, a back, a first side and a second side; the movable connector having a first axis extending between the first end and the second end, a second axis extending between the front and the back, and a third axis extending between the first side and the second side; the movable connector having an initial state with a memory, and being movable in at least two of the first axis, the second axis and the third axis.

5. The transducer array of claim 1, wherein the movable connector is a micro spring.

6. The transducer array of claim 5, wherein the micro spring has an inner spring diameter in the range of about 0.1 mm to 3.2 mm.

7. The transducer array of claim 1, wherein the movable connector is a spring contact.

8. The transducer array of claim 7, wherein the spring contact has a height of about 1.0 mm to 2.0 mm.

9. The transducer array of claim 1, wherein the electrically conductive layer comprises a plurality of spatially disposed electrode elements.

10. The transducer array of claim 9, wherein each of the spatially disposed electrode elements has a first surface and a second surface opposite the first surface, the first surface in contact with the movable connector, and wherein the transducer array comprises a dielectric layer positioned adjacent to the second surface of the electrically conductive layer.

11. The transducer array of claim 10, wherein the dielectric layer comprises a plurality of spatially disposed dielectric elements positioned adjacent to the second surface of respective electrode elements.

12. The transducer array of claim 11, wherein the dielectric elements include a flexible polymer material.

13. The transducer array of claim 11, wherein the dielectric elements include a ceramic material.

14. The transducer array of claim 1, wherein the conductive skin adjacent layer comprises conductive gel.

15. The transducer array of claim 1, wherein the conductive skin adjacent layer comprises a conductive adhesive.

16. The transducer array of claim 1, wherein the electrode assembly further comprises a compression layer operable to apply a force upon the movable connector, causing the movable connector to move relative to the substrate when the transducer array is placed upon the skin of a patient.

17. A system for delivering TTFields to a body of a subject, the system comprising:
an electric field generator configured to generate an electrical signal having an alternating current waveform at a frequency in a range from 50 kHz to 1 MHz;
a first conductive lead electrically coupled to the electric field generator, the first conductive lead configured to carry the electrical signal;
a first transducer array coupled to the first conductive lead, the first transducer array having a first electrode assembly comprising a first substrate, a first electrically conductive layer, a first movable connector extending between the first substrate and the first electrically conductive layer, and electrically connected to the first conductive lead, and a first conductive skin adjacent layer, the first movable connector constructed of a conductive material operable to carry the electrical signal to the first electrically conductive layer and to maintain the first electrically conductive layer parallel to a patient's skin when the first transducer array is applied to the patient's skin;
a second conductive lead electrically coupled to the electric field generator, the second conductive lead configured to carry the electrical signal; and
a second transducer array coupled to the second conductive lead, the second transducer array receiving the electrical signal from the second conductive lead, the second transducer array comprising a second electrode assembly connected to a second conductive skin adjacent layer.

18. The system of claim 17, wherein the first movable connector is movable so as to enable movement of the electrically conductive layer relative to the first substrate so as to position the first electrically conductive layer parallel to a patient's skin when the first substrate is not parallel to the patient's skin.

19. A method, comprising:
applying at least two conductive regions to a skin of a patient;
coupling the conductive regions to an electric field generator, the electric field generator configured to generate an electrical signal having an alternating current waveform at a frequency in a range from 50 kHz to 1 MHz, each conductive region having an electrode assembly comprising an electrically conductive layer and an electrically conductive movable connector extending between the electrically conductive layer and a substrate, and a conductive skin adjacent layer attached to the electrically conductive layer, the movable connector being operable to supply the electrical signal to the electrically conductive layer and to maintain the electrically conductive layer parallel to the skin, the electrically conductive layer being electrically coupled with a patient's skin; and
activating the electric field generator to supply the electrical signal to the conductive regions, thereby supplying electrical current to the patient through the conductive skin adjacent layer.

20. The method of claim 19, wherein the movable connector is movable so as to enable movement of the electrically conductive layer relative to the substrate so as to position the electrically conductive layer parallel to a patient's skin when the substrate is not parallel to the patient's skin, and wherein the step of applying the at least two conductive regions to the skin of the patient includes applying the conductive regions so that the electrically conductive layer is parallel to a patient's skin and the substrate is not parallel to the patient's skin.
